(19) United States
(12) Patent Application Publication
(10) Pub. No.: US 2023/0330054 A1
(43) Pub. Date: Oct. 19, 2023

(54) METHOD FOR PREVENTING OR TREATING LUNG INFECTION AND LUNG INFLAMMATION

(71) Applicant: Olatec Therapeutics LLC, New York, NY (US)

(72) Inventors: Charles A. Dinarello, Boulder, CO (US); Damaris B. Skouras, New York, NY (US)

(21) Appl. No.: 17/995,167

(22) PCT Filed: Mar. 26, 2021
(86) PCT No.: PCT/US2021/024465

§ 371 Date: Sep. 30, 2022

Related U.S. Application Data
(60) Provisional application No. 63/003,075, filed on Mar. 31, 2020.
(60) Provisional application No. 63/122,274, filed on Dec. 7, 2020.

(51) Int. Cl. A61K 31/275; A61K 9/00

(52) U.S. Cl. CPC A61K 31/275; A61K 9/0053

(57) ABSTRACT

The present invention is directed to a method for preventing or treating lung infection and/or lung inflammation. The present invention provides a method for preventing or treating macrophage activation syndrome in an infected patient. The present invention also provides a method preventing or treating pneumonitis. The present invention further provides a method for treating COVID-19 patients with SAR-CoV-2 viral infection having mild or moderate respiratory symptoms or fever. The method comprises administering to a subject in need thereof dapansutrile, or a pharmaceutically acceptable solvate thereof, in an effective amount. The present method treats early cytokine release syndrome and arrests the initiation of the IL-1β and IL-18 mediated “cytokine storm” and/or pneumonitis in a patient, without causing a negative effect to his heart condition, type II diabetes, and other issues. Oral administration is a preferred route of administration.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence Listing.txt with a creation date of Mar. 24, 2021 and a size of 1.50 kilobytes. The Sequence Listing filed via EFS-Web is part of the specification and is hereby incorporated in its entirety by reference herein.

### FIELD OF THE INVENTION

The present invention relates to using dapansutrile, or a pharmaceutically acceptable solvate thereof, for preventing or treating lung infection and/or lung inflammation. The present invention is useful in preventing or treating macrophage activation syndrome in an infected patient. The present invention is also useful for preventing or treating pneumonitis. The prevent invention is further useful in treating COVID-19 patients with mild or moderate respiratory symptoms.

### BACKGROUND

Macrophage activation syndrome (MAS) is a severe complication of rheumatic disease in childhood, particularly in systemic juvenile idiopathic arthritis. MAS is characterized by pancytopenia, liver insufficiency, coagulopathy, and neurologic symptoms and is caused by uncontrolled activation and proliferation of T lymphocytes and well-differentiated macrophages, leading to widespread hemophagocytosis and cytokine overproduction.

MAS is severe inflammation of the immune system and is a very serious condition. MAS is usually associated with known rheumatologic conditions, infections, viruses and cancers.

The hallmark clinical and laboratory features include high fever, hepatosplenomegaly, lymphadenopathy, pancytopenia, liver dysfunction, disseminated intravascular coagulation, hypofibrinogenemia, hyperferritinemia, and hypertriglyceridemia. Despite marked systemic inflammation, the erythrocyte sedimentation rate (ESR) is paradoxically depressed, caused by low fibrinogen levels. The low ESR helps to distinguish the disorder from a flare of the underlying rheumatic disorder, in which case the ESR is usually elevated. A bone marrow biopsy or aspirate usually shows hemophagocytosis.

Macrophage activation syndrome, also known as secondary hemophagocytic lymphohistiocytosis, is classically defined by the presence of 5 of 8 clinical criteria including Ferritin >500 ng/ml, two-line cytopenia, organomegaly, hyper-triglyceridemia, hypofibrinogenemia, elevated sCD25, absent NK cytotoxic activity, and hemophagocytosis.

MAS commonly develops after viral infections and characteristically has high D-dimer and circulating IL-18. A severe IL-18/IL-18BP imbalance results in T helper 1 (Th-1) lymphocyte and macrophage activation, which escapes control by NK-cell cytotoxicity and may allow for secondary hemophagocytic syndrome in patients with underlying diseases. (Mazodier, K., et al., (2005), Blood 106, 3483-3489).

There is a public health crisis threatening the world with the emergence and spread of novel coronaviruses—including SARS-CoV-2, the virus responsible for COVID-19 disease. Coronavirus disease 2019 (COVID-19) is primarily a respiratory disease characterized by fever, cough, and shortness of breath, caused by a new strain of coronavirus (severe acute respiratory syndrome coronavirus 2 [SARS-CoV-2]); in some infected subjects, COVID-19 has manifestations of systemic organ involvement. While the majority of individuals diagnosed with COVID-19 experience mild symptoms, others may progress quickly to acute respiratory stress and multi-organ failure. The relentless progression of COVID-19 is due in part to the absence of proven therapeutic interventions beyond supportive care and respiratory support, both of which have demonstrated limited benefit or availability.

The lungs are typically the organs first and most affected by COVID-19 because the virus accesses host cells via the enzyme ACE2 (angiotensin-converting enzyme2), which is most abundant in the type II alveolar cells of the lungs. COVID-19 viruses use peplomers (glycoprotein spikes) on the viral capsid to connect to ACE2 and enter the host cell.

There is a need for an effective method for preventing or treating MAS in an infected patient. There is also a need for a method for treating COVID-19 patient with mild or moderate respiratory symptoms; the method should reduce: (1) hospitalization (if treated on an outpatient basis), (2) ventilation or intubation (3) ARDS and (4) mortality of the patient.

### BRIEF DESCRIPTION OF THE DRAWINGS

FIGS. 1A-1F show increased biomarkers in early COVID-19 patients. Mean±SEM of plasma IL-1β (1A), IL-6 (1B), IL-1Ra (1C), TNF α (1D), IL-10 (1E) and uPAR (urokinase plasminogen activator receptor, 1F) in SARS-CoV-2 positive patients (N=39) are compared to SARS-CoV-2 negative (N=24). *p<0.05; **p<0.01; ***p<0.001; ****p<0.0001.

FIGS. 2A-2D show increased NLRP3 levels in early COVID-19 patients. FIGS. 2A and 2B show fold change of NLRP3 (2A) and IL-1β (2B) mRNA levels from buffy coats of SARS-CoV-2 positive patients (N=27) compared to SARS-CoV-2 negative (N=14) (Wilcoxon signed-rank test). FIG. 2C shows fold change of caspase-1 mRNA levels from buffy coats of SARS-CoV-2 positive patients (N=27) compared to SARS-CoV-2 negative (N=14) (Wilcoxon signed-rank test). FIG. 2D shows NLP3 protein levels in monocytes isolated from SARS-CoV-2 positive patients (N=2) compared to SARS-CoV-2 negative patients (N=1). *p<0.05

FIG. 3 is a flow chart showing the processes of primary lung infection and lung inflammation and the circulation of cytokines.

FIGS. 4-1 to 4-2 show the study design of clinical trial (Example 4) to evaluate the safety and efficacy of orally administered dapansutrile capsules for treating COVID-19 patients with mild or moderate respiratory symptoms on an outpatient basis.

FIGS. 5-1 to 5-3 show the study design of clinical trial to evaluate the safety and efficacy of orally administered dapansutrile capsules for treating COVID-19 patients with mild to moderate COVID-19 symptoms.

### DETAILED DESCRIPTION OF THE INVENTION

Following entry and replication of Severe Acute Respiratory Syndrome-coronavirus 2 (SARS-CoV-2) into ACE2 expressing cells, the infected cells undergo lysis releasing more virus but also cell contents. In the lung, cytokines such as IL-1β are released together with other cell contents. A cascade of inflammatory cytokines ensues, including chemokines and IL-1β, triggering both local as well as systemic inflammation. This cascade of inflammatory cytokines in patients with COVID 19 is termed “Cytokine Release Syndrome” (CRS), and is associated with poor outcomes and death.

The present invention is directed to a method for preventing or treating lung infection and lung inflammation. The present invention is useful in treating macrophage activation syndrome in an infected patient, e.g., in a viral-infected patient. The present invention is useful for treating COVID-19 patients with mild or moderate respiratory symptoms. The present invention is useful in treating COVID-19 subjects presenting with mild to moderate COVID-19 symptoms and evidence of early cytokine release syndrome. The present invention is further useful in treating pneumonitis. The method comprises administering to a subject in need thereof an effective amount of dapansutrile, or a pharmaceutically acceptable solvate thereof.

### Compound

The present invention uses a purified compound of dapansutrile (3-methanesulfonylpropionitrile), or a pharmaceutically acceptable solvate thereof:

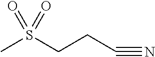

“Pharmaceutically acceptable solvates,” as used herein, are solvates that retain the desired biological activity of the parent compound and do not impart undesired toxicological effects. Solvates are addition complexes in which the compound is combined with an acceptable co-solvent in some fixed proportion. Co-solvents include, but are not limited to, water, ethyl acetate, lauryl lactate, myristyl lactate, cetyl lactate, isopropyl myristate, methanol, ethanol, 1-propanol, isopropanol, 1-butanol, isobutanol, tert-butanol, acetone, methyl ethyl ketone, acetonitrile, benzene, toulene, xylene(s), ethylene glycol, dichloromethane, 1,2-dichloroethane, N-methylformamide, N,N-dimethylformamide, N-methylacetamide, pyridine, dioxane, and diethyl ether.

Dapansutrile, is a small molecule that selectively inhibits the nucleotide-binding and oligomerization domain (NOD)-like receptor pyrin domain protein 3 (NLRP3) inflammasome which in turn prevents the activation of caspase-1 and the maturation of pro-interleukin-1 β (proIL-1β) and pro-interleukin-18 (pro-IL-18) to the pro-inflammatory cytokines IL-1β and IL-18, respectively (Marchetti, C., et al (2018). Proc Natl Acad Sci USA 115, E1530-E1539).

### Pharmaceutical Compositions

The present invention provides pharmaceutical compositions comprising one or more pharmaceutically acceptable carriers and an active compound of dapansutrile, or a pharmaceutically acceptable salt, or a solvate thereof. The active compound or its pharmaceutically acceptable salt or solvate in the pharmaceutical compositions in general is in an amount of about 1-90% for a tablet formulation; about 1-100% for a capsule formulation; about 0.01-20%, or 0.05-20%, or 0.1-20%, or 0.2-15%, or 0.5-10%, or 1-5% (w/w), for a topical formulation; about 0.1-5% for an injectable formulation, and 0.1-5% for a patch formulation. The active compound used in the pharmaceutical composition in general is at least 90%, preferably 95%, or 98%, or 99% (w/w) pure.

In one embodiment, the pharmaceutical composition is in a dosage form such as tablets, capsules, granules, fine granules, powders, syrups, suppositories, injectable solutions, patches, or the like. In another embodiment, the active compound is incorporated into any acceptable carrier, including creams, gels, lotions or other types of suspensions that can stabilize the active compound and deliver it to the affected area by topical applications. The above pharmaceutical composition can be prepared by conventional methods.

Pharmaceutically acceptable carriers, which are inactive ingredients, can be selected by those skilled in the art using conventional criteria. Pharmaceutically acceptable carriers include, but are not limited to, non-aqueous based solutions, suspensions, emulsions, microemulsions, micellar solutions, gels, and ointments. The pharmaceutically acceptable carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, phosphate, citrate, acetate, borate; and trolamine; antioxidants such as salts, acids and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants such as lecithin, phospholipids, including but not limited to phosphatidylcholine, phosphatidylethanolamine and phosphatidyl inositiol; poloxamers and poloxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and hydrophobically modified cross-linked acrylate copolymer; polysaccharides such as dextrans and glycosaminoglycans such as sodium hyaluronate. Such pharmaceutically acceptable carriers may be preserved against bacterial contamination using well-known preservatives, these include, but are not limited to, benzalkonium chloride, ethylenediaminetetraacetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet formulation or a capsule formulation of the active compound may contain other excipients that have no bioactivity and no reaction with the active compound. Excipients of a tablet or a capsule may include fillers, binders, lubricants and glidants, disintegrators, wetting agents, and release rate modifiers. Binders promote the adhesion of particles of the formulation and are important for a tablet formulation. Examples of binders include, but not limited to, carboxymethylcellulose, cellulose, ethylcellulose, hydroxypropylmethylcellulose, methylcellulose, karaya gum, starch, starch, and tragacanth gum, poly(acrylic acid), and polyvinylpyrrolidone.

For example, a patch formulation of the active compound may comprise some inactive ingredients such as 1,3-butylene glycol, dihydroxyaluminum aminoacetate, disodium edetate, D-sorbitol, gelatin, kaolin, methylparaben, polysorbate 80, povidone (polyvinylpyrrolidone), propylene glycol, propylparaben, sodium carboxymethylcellulose, sodium polyacrylate, tartaric acid, titanium dioxide, and purified water. A patch formulation may also contain skin permeability enhancer such as lactate esters (e.g., lauryl lactate) or diethylene glycol monoethyl ether.

Topical formulations including the active compound can be in a form of gel, cream, lotion, liquid, emulsion, ointment, spray, solution, and suspension. The inactive ingredients in the topical formulations for example include, but not limited to, lauryl lactate (emollient/permeation enhancer), diethylene glycol monoethyl ether (emollient/permeation enhancer), DMSO (solubility enhancer), silicone elastomer (rheology/texture modifier), caprylic/capric triglyceride, (emollient), octisalate, (emollient/UV filter), silicone fluid (emollient/diluent), squalene (emollient), sunflower oil (emollient), and silicone dioxide (thickening agent).

### Method of Use

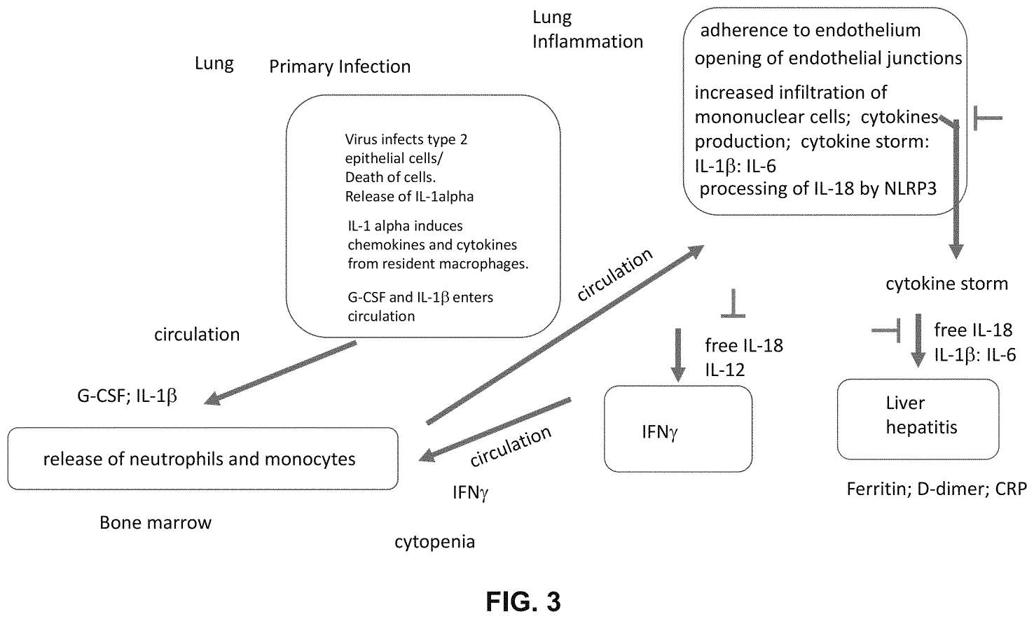

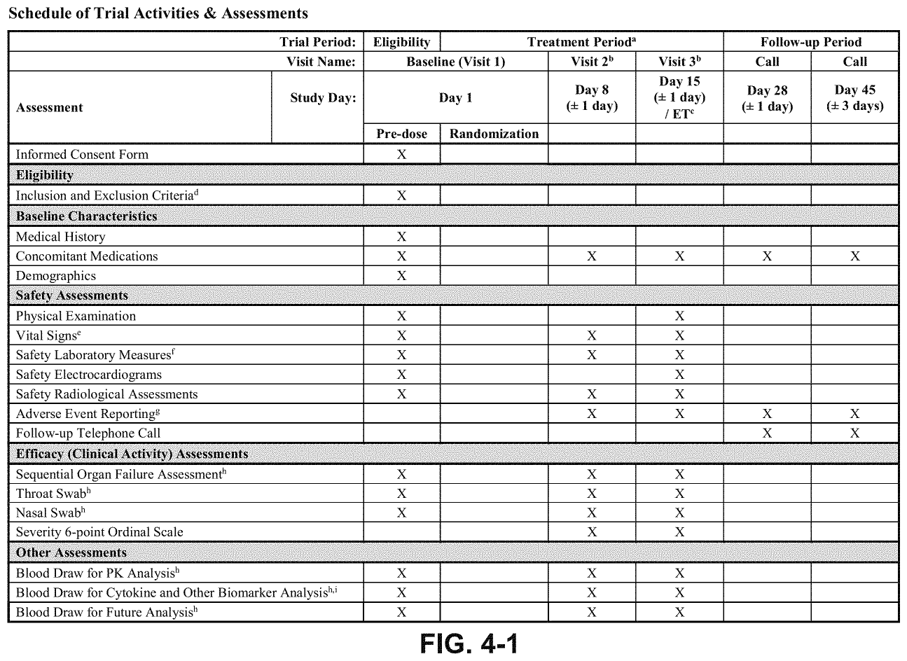

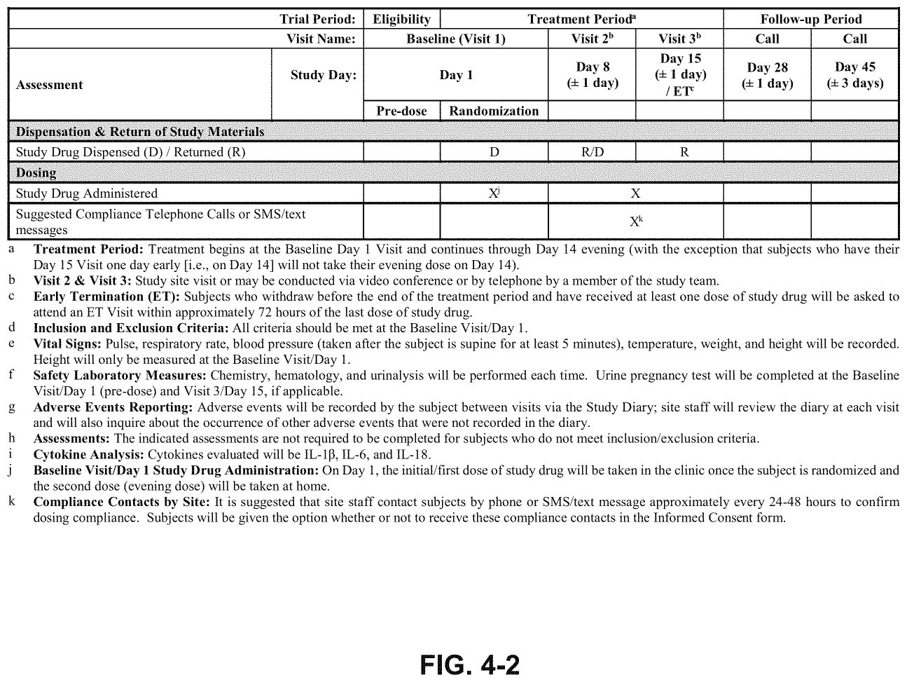

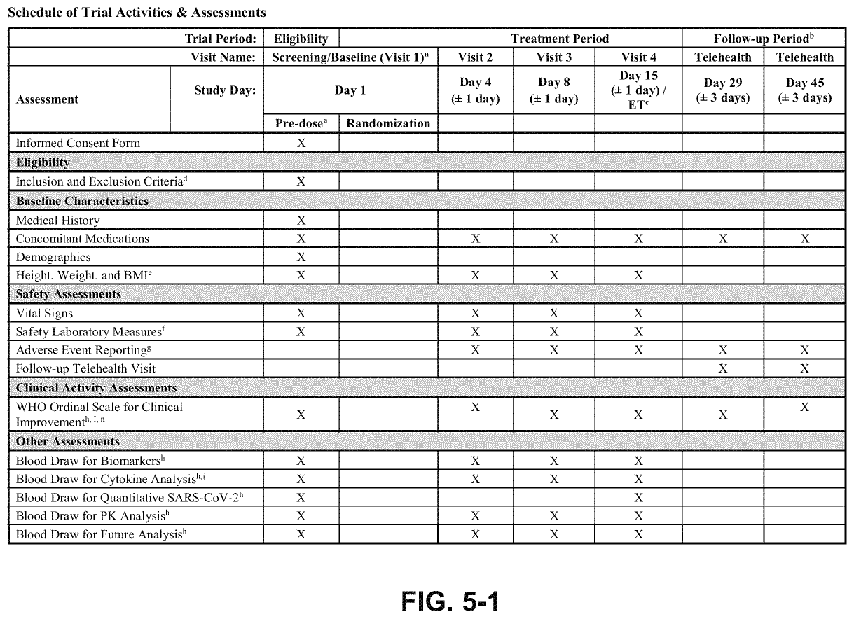

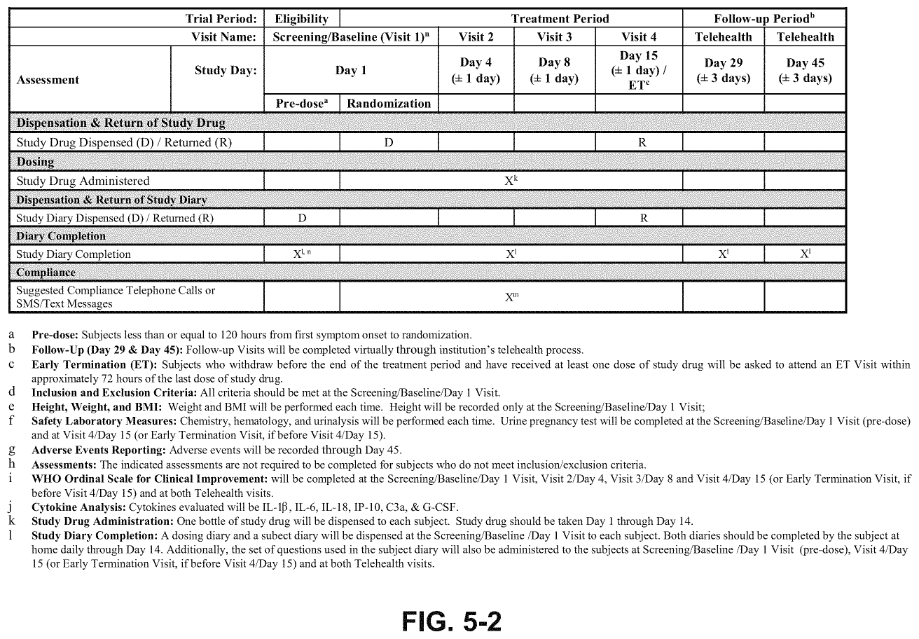

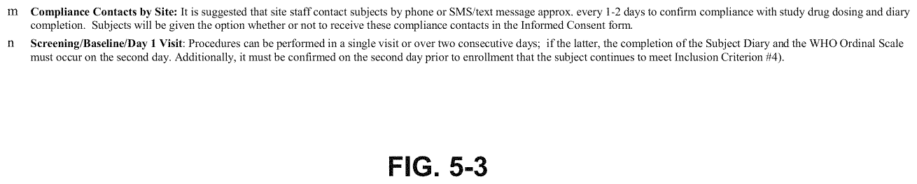

The inventors summarize the processes of primary lung infection and lung inflammation and the circulation of cytokines in a flow chart (FIG. 3).

In a first aspect, the present invention is directed to a method for preventing or treating macrophage activation syndrome in an infected patient, e.g., a viral infected patient. The method comprises the steps of first identifying a subject who is prone to develop MAS or who suffers from MAS and administering to the subject an effective amount of dapansutrile. “An effective amount,” as used herein, is the amount effective to prevent or to treat a disease by ameliorating the pathological condition or reducing the symptoms of MAS. For example, an effective amount for treating MAS ameliorates one or more pathological conditions or symptoms of high fever, hepatosplenomegaly, lymphadenopathy, pancytopenia, liver dysfunction, disseminated intravascular coagulation, hypofibrinogenemia, hyperferritinemia, and hypertriglyceridemia.

In one embodiment, the patient has an underlying disease of chronic obstructive pulmonary disease (COPD), diabetes, and/or heart disease.

In another embodiment, the patient is a COVID-19 patient who is infected with the SARS-CoV-2 virus.

MAS commonly develops after viral infections and is characterized by having elevated D-dimer and circulating IL-18. Dapansutrile reduces IL-1β and IL-18, and may further reduce high D-dimer. Dapansutrile inhibits IL-1β-mediated auto-inflammation and reduces the infiltration of macrophages and neutrophils into the lungs. Dapansutrile is effective in treating inflammation, e.g., early cytokine release syndrome and treating the early stages of MAS caused by viral infection, and is effective in preventing cytokine storm, by reducing reduces IL-1β and IL-18. In MAS, heart failure can result due to IL-18 and other upregulated cytokines. By treating the early stages of MAS in a patient, dapansutrile further prevents heart failure in the patient.

In a second aspect, the present invention is directed to a method for treating a COVID-19 patient, either in an early stage or in a late stage. The present invention is particularly effective in treating COVID-19 patient with mild or moderate respiratory symptoms, either on an inpatient or outpatient basis. The method comprises the steps of first identifying a patient who suffers from COVID-19 infected with the SARS-CoV-2 virus and has mild to moderate respiratory symptoms, and then administering to the subject an effective amount of dapansutrile.

In another aspect, the present invention provides a method to treat patients infected with SARS CoV 2 early in the course of the disease by administering to patients dapansutrile, which is a specific NLRP3 inhibitor, in order to arrest the progression of IL-1β mediated CRS. Such a treatment offers an opportunity to reduce hospitalization and the need for supplemental oxygen, particularly in subjects with high risk co morbidities.

The morbidity and mortality of COVID 19 often takes place when SARS CoV 2 RNA is absent in secretions in patients as disease worsens and is associated with marked increases in biomarkers of the CRS. Thus, the CRS in COVID-19 is indicative of the destructive properties of IL-1β and its downstream cytokines in the lung. The present method treats patients with dapansutrile and reduces the detrimental properties of IL-1β and its downstream cytokines by first preventing the processing and release of active IL-1β.

There are two mechanisms that dapansutrile treats COVID-19 patients. First, dapansutrile inhibits IL-1β-mediated auto-inflammation by reducing the monocyte processing of the IL-1β precursor and reducing the infiltration of macrophages and neutrophils into the lungs. Second, dapansutrile directly inhibits NLRP3 activation by COVID-19 by blockade of apoptosis-associated speck-like protein containing a caspase recruitment domain (ASC) as was seen in HEK293 cells expressing ACE2, which functions for viral entry. Dapansutrile reduces inflammation caused by virus-induced IL-1β release from resident macrophages in the lung or monocytes that have infiltrated into the lung from the bone marrow. Dapansutrile also significantly reduces circulating IL-6.

By preventing the down-stream IL-1β and IL-18 mediated cytokine storm and/or pneumonitis, the present method reduces COVID-19 mediated inflammation and prevents the progression of COVID-19 disease to critical stages, i.e., the present method reduces the progression of lung inflammation, the need for ventilation and intubation, and mortality of the patient. Dapansutrile specifically inhibits NLRP3, reduces both IL-1β and IL-18, and thus targets two agonists of COVID 19 disease.

In one embodiment, the present invention is useful in treating early-stage COVID-19 patients who are not hospitalized. These patients may take dapansutrile orally at home, which decreases patients' susceptibility to hospital-borne infection.

In one embodiment, the patient has mild respiratory symptoms (fever, cough, mild to moderate dyspenea), but is pre-pneumonitis, and the method prevents cytokine storm and/or pneumonitis.

In one embodiment, the patient has moderate COVID-19 symptoms; i.e., the patient has fever (temperature ≥38° C./100.4° F.) and shortness of breath (with exertion); the patient does not require oxygen, and meets the definition of “moderate” as set forth by the May 2020 FDA Guidance for Industry: COVID-19: Developing Drugs and Biological Products for Treatment or Prevention (FDA, 2020), which includes all of the following criteria: a. respiratory rate: ≥20 breaths/minute, b. SpO₂: >93% on room air at sea level, and c. Heart rate: ≥90 beats/minute.

In one embodiment, the method reduces the progression of lung inflammation and the progression to acute respiratory distress syndrome, ARDS, in the patient.

In one embodiment, the method reduces sequential organ failure in cardiovascular, respiratory, hepatic, coagulation, renal, and/or neurological systems.

In one embodiment, the method prevents a patient from hospitalization if being treated with dapansutrile on an outpatient basis.

In one embodiment, the method reduces the needs of a patient for supplemental oxygen such as non-invasive ventilation, high flow oxygen device, invasive mechanical ventilation, or extracorporeal membrane oxygenation (ECMO).

In one embodiment, the method reduces the hospitalization rate and mortality rate of patients.

In one embodiment, the method exacerbates the high-risk conditions/comorbidities in diabetes, uncontrolled hypertension, a respiratory disease, heart failure, and a coronary disease.

In one embodiment, the method reduces residual fever, headaches, loss of taste, and/or loss of smell that lingers after the COVID-19 disease.

In one embodiment, the method mitigates the pulmonary and systemic sequelae associated with early cytokine release syndrome in coronavirus disease, such as COVID-19.

The pharmaceutical composition of the present invention can be applied by systemic administration and local administration. Systemic administration includes oral, parenteral (such as intravenous, intramuscular, subcutaneous or rectal), and other systemic routes of administration. In systemic administration, the active compound first reaches plasma and then distributes into target tissues. Local administration includes topical administration.

Dosing of the composition can vary based on the extent of the disease and each patient's individual response. For systemic administration, plasma concentrations of the active compound delivered can vary; but are generally 0.1-1000 μg/mL or 1-100 μg/mL.

In one embodiment, the pharmaceutical composition is administrated orally to the subject. The dosage for oral administration is generally at least 1 mg/kg/day and less than 100 mg/kg/day. For example, the dosage for oral administration is 1-100, or 5-50, or 10-50 mg/kg/day, for a human subject. For example, the dosage for oral administration is 100-10,000 mg/day, and preferably 500-2000, 500-4000, 500-4000, 1000-5000, 2000-5000, 2000-6000, or 2000-8000 mg/day for a human subject. The drug can be orally taken once, twice, three times, or four times a day. The patient is treated daily for 14 days up to 1 month, 2 months, or 3 months or for lifespan.

In one embodiment, the pharmaceutical composition is administrated intravenously to the subject. The dosage for intravenous bolus injection or intravenous infusion is generally 0.03 to 20 or 0.03 to 10 mg/kg/day.

In one embodiment, the pharmaceutical composition is administrated subcutaneously to the subject. The dosage for subcutaneous administration is generally 0.3-20 or 0.3-3 mg/kg/day.

Those of skill in the art will recognize that a wide variety of delivery mechanisms are also suitable for the present invention.

The present invention may be used in combination with one or more other treatments that treat COVID-19.

The present invention is useful in treating a mammal subject or a mammal patient. The present invention is particularly useful in treating humans. A “subject” and a “patient” are used interchangeably in the application.

The following examples further illustrate the present invention. These examples are intended merely to be illustrative of the present invention and are not to be construed as being limiting.

### EXAMPLES

### Example 1. Methods

The following methods were used to generate the results of Examples 2 and 3.

### PBMCs

Peripheral blood mononuclear cells (PBMCs) were isolated from drawn blood by gradient centrifugation using Ficoll-Paque (Pharmacia Biotech, Uppsala, Sweden). PBMCs were suspended in Roswell Park Memorial Institute 1640 medium supplemented with 50 mg/mL gentamicin, 2 mM %-glutamine, and 1 mM pyruvate and cultured for 24 hours.

### Cytokines Measurements

Plasma levels of IL 1□, IL 6, IL 10 and TNF□ were measured with the Ella platform (Protein Simple, San Jose, CA, USA) using multiplex cartridges. Soluble urokinase plasminogen activator receptor (uPAR) was determined using Quantikine kits (R&D Systems, Minneapolis, MI, USA).

### Gene Expression

RNA was isolated according to the manufacturer's protocol (Thermo Fisher Scientific) and synthesized into cDNA using SuperScript III First-Strand (Thermo Fisher Scientific). Quantitative PCR (qPCR) was performed on cDNA using Power SYBR Green PCR master mix (Thermo Fisher Scientific) on Biorad CFX96 Real time system. Gene expression was carried for the following mRNAs: nlrp3, caspase1 and il1b with gapdh used as reference gene, using the following primers:

nlrp3:
(SEQ ID NO: 1)
GAATCTCAGGCACCTTTACC
and
(SEQ ID NO: 2)
GCAGTTGTCTAATTCCAACACC
caspase1:
(SEQ ID NO: 3)
AAGTCGGCAGAGATTTATCCA
and
(SEQ ID NO: 4)
GATGTCAACCTCAGCTCCAG
il1b:
(SEQ ID NO: 5)
CTAAACAGATGAAGTGCTCCTTCC
and
(SEQ ID NO: 6)
CACATAAGCCTCGTTATCCCA

### Western Blotting.

Cells were lysed using RIPA buffer supplemented with protease inhibitors (Roche), centrifuged at 13,000 g for 20 min at 4° C. and the supernatants were obtained. Protein concentration was determined in the clarified supernatant using Bio-Rad protein assay (Bio-Rad Laboratories, Hercules, CA). Proteins were electrophoresed on Mini-PROTEAN TGX 4-20% gels (Bio-Rad Laboratories) and transferred to nitrocellulose 0.2 μM (GE Water & Process Technologies). Membranes were blocked in 5% dried milk in PBS-T 0.5% for 1 hour at room temperature. Primary antibodies for NLRP3 1:1000 (Adipogen) was used in combination with peroxidase-conjugated secondary antibodies and chemiluminescence to detect the protein concentration. A primary antibody against β-actin (Santa Cruz Biotechnology) was used to assess protein loading.

### Statistical Analysis

Significance of differences was evaluated with Student's two-tail T-test using GraphPad Prism (GraphPad Software Inc, La Jolla, CA) or Wilcoxon signed-rank test as indicated. For the correlation studies the distribution were computed using Pearson correlation coefficients and Statistical significance was calculated with two-tailed option with the confident interval set at 95%. Statistical significance was set at p<0.05.

### Example 2. Increased Circulating IL-1β Level in Covid-19 Patients

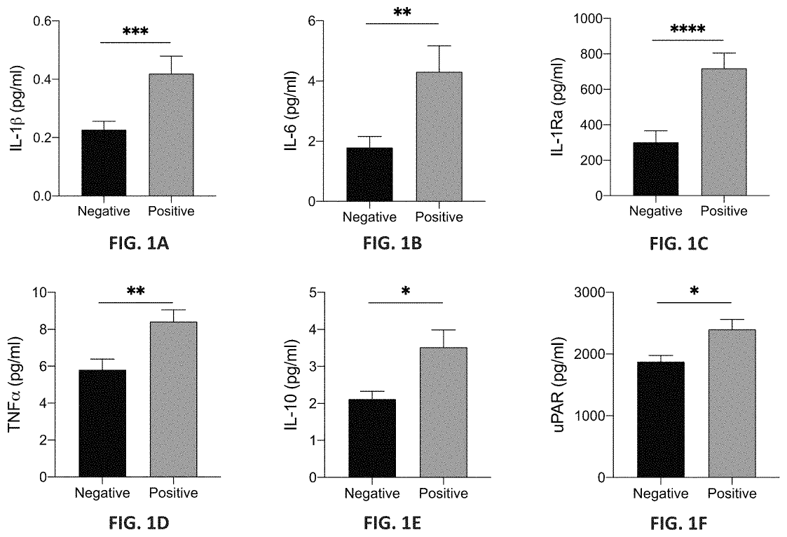

As shown in FIG. 1A, compared to healthy controls, circulating IL-1β is elevated in ambulatory subjects positive for SARS CoV 2. The mean level of IL-1β in 39 healthy subjects is 0.23 pg/mL±0.03, whereas in 24 Covid-19 subjects have the mean IL-1β level is 2 fold greater (0.42±0.06 pg/mL; p<0.001). Similarly, mean IL 6 level in infected subjects is greater than 2-fold higher (FIG. 1B, p<0.01). The naturally occurring IL 1 Receptor antagonist (IL 1Ra) is 2.5-fold higher in Covid-19 positive individuals compared to healthy subjects (FIG. 1C, p<0.0001). Also shown in FIG. 1D-1F are significantly elevated levels of tumor necrosis factor alpha (TNFα), IL 10 and urokinase plasminogen activator receptor (uPAR).

### Example 3. Increased NLRP3 Level in Covid-19 Patients

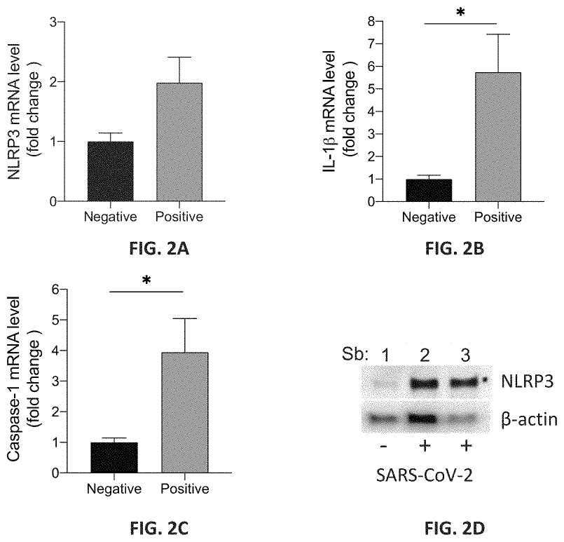

FIG. 2A shows a 2 fold increase in NLRP3 levels in buffy coat cells from 27 Covid-19 positive subjects compared to cells from 14 negative subjects. In the same cells, there was a 5.5 fold increase in IL-1β gene expression (FIG. 2B). As shown in FIG. 2C, Caspase 1 gene expression is elevated 4 fold in Covid-19 positive subject. Thus, in COVID-19 patients the molecular cascade resulting in elevated circulating IL-1β and NLRP3 gene expression is initiated with the infection, critical for disease development is the release of active IL-1β from its inactive precursor to processing and release of active IL-1β. Using Western blotting we show evidence of the NLRP3 protein in monocytes from two infected subjects (FIG. 2D), but not in cells from an uninfected subject.

Examples 2-3 demonstrate highly significant circulating levels of IL-1β, IL 1 Receptor antagonist (IL-1Ra), IL 6, TNFα, IL-10 and soluble urokinase plasminogen activator receptor (uPAR) in COVID 19 patients with mild or no symptoms. The results also show that in circulating myeloid cells from the same patients, there was increased expression of the NOD-, LRR- and pyrin domain-containing 3 (NLRP3) receptor early in the infection. We observed that the increase in NLRP3 gene expression correlated with IL-1β gene expression and with elevated circulating IL-1β levels. These observations establish that early in SARS CoV 2 infection, NLRP3 activation takes place and initiates the CRS. Thus, NLRP3 is a target to reduce the tissue damage of inflammatory cytokines of the CRS.

### Example 4. Clinical Study of Orally Administered Dapansutrile Capsules in COVID-19 Patients with Mild or Moderate Respiratory Symptoms

### Methodology

The study is a randomized, double-blinded, placebo-controlled, multi-center trial to evaluate safety and efficacy of orally administered dapansutrile capsules for treating COVID-19 patients with mild to moderate respiratory symptoms on an outpatient or inpatient basis.

### Main Criteria for Inclusion:

1) Male and female subjects between 18 and 80 years old, inclusive;
2) Laboratory-confirmed diagnosis of COVID-19 disease, testing positive for the SARS-CoV-2 viral infection as determined by polymerase chain reaction (PCR) or other commercial or public health assay in any specimen <72 hours prior to randomization;
3) Cough, fever>38.5° C.;
4) Mild or moderate respiratory symptoms defined as SpO₂>94% on room air
5) Mild to moderate dyspnea (SpO₂>94% measured with a peripheral pulse oximeter and respiratory rate between 20 to 30 breaths without stridor;
6) Radiographic infiltrates by imaging (chest X-ray, CT scan, etc.), or clinical assessment (no evidence of rales/crackles on exam) and SpO₂>94% on room air;
7) Acceptable overall medical condition to be safely enrolled in and to complete the study in the opinion of the Investigator;
8) Ability to provide written, informed consent prior to initiation of any study-related procedures, and ability, in the opinion of the Investigator, to understand and comply with all the requirements of the study, which includes abstaining from use of use of prohibited medications.

### Main Criteria for Exclusion:

1) Women of childbearing potential, or men whose sexual partner(s) is a woman of childbearing potential, who:
a. Are or intend to become pregnant during the study,
b. Are nursing,
c. Are not using an acceptable, highly effective method of contraception until all follow-up procedures are complete.
2) Participation in any other clinical trial of an experimental treatment for COVID-19.
3) Peripheral capillary oxygen saturation (SpO₂)<94% on room air at sea level at screening.
4) Requiring mechanical ventilation and/or supplemental oxygen.
5) Evidence of multiorgan failure.
6) Use of any prohibited concomitant medications/therapies including:
a. Use of strong or moderate CYP3A4 inhibitors (such as diltiazem, verapamil, etc.) or P-gp inhibitors within the prior 14 days to the Baseline Visit/Day 1,
b. Use of cyclooxygenase (COX) inhibitors.
7) Known history of renal impairment (e.g., calculated glomerular filtration rate [GFR]<45 mL/min).
8) Any other concomitant medical or psychiatric conditions, diseases, or prior surgeries that, in the opinion of the Investigator, would impair the subject from safely participating in the trial and/or completing protocol requirements.
9) Enrollment in any trial and/or use of any investigational medicinal product or device within the immediate 30-day period prior to the Baseline.

### Dose and Mode of Administration:

The study design of Example 1 is shown in FIG. 2 for outpatients or ambulatory care. The study design for inpatients or hospitalized patients is similar to that described in FIG. 2 except patients are confined in the hospital and visits are all on-site visits.

Subjects are randomly assigned and blinded to receiving either 2000 mg/day dapansutrile or placebo. Each cohort consists of 40 patients; 40 patients are treated with dapansutrile and 40 patients are treated with placebo or active control.

### Clinical Trial Duration:

The trial duration is approximately 45 days for all subjects enrolled, with 3 visits to the study site or phone calls to each subject in lieu of a site visit: Baseline Visit/Day 1, Visit 2/Day 8 (±1 day), Visit 3/Day 15 (±1 day), and a follow-up telephone contact on Day 28 and Day 45 (±3 days).

### Clinical Activity Outcomes for Evaluation:

### Primary Clinical Activity Outcome Measure:

The response rate of dapansutrile as compared to placebo at Day 15. Response is defined as not progressing to pneumonia or ventilation by Day 15.

### Principle Secondary Clinical Activity Outcome Measures:

Change of SOFA (Sequential Organ Failure Assessment) at Day 15 evaluating 6 variables, each representing an organ system (one for respiratory, cardiovascular, hepatic, coagulation, renal and neurological systems) and scored from 0 (normal) to 4 (high degree of dysfunction/failure. Maximum score ranges from 0-24;
Change from Baseline to Day 15 radiologic response (thoracic CT scan or chest x-ray);
Change from Baseline to Day 15 in respiratory symptoms;
Change from Baseline to Day 15 in peripheral blood O₂ saturation;
Change from Baseline to Day 15 in dyspnea (on a scale of absent, mild, moderate, and severe);
Change from Baseline to Day 15 in fever, cough, myalgia, respiratory symptoms, SaO₂ ambient air;
Change from Baseline to Day 15 in plasma levels of C-reactive protein (CRP) and inflammatory cytokines (IL-1β, IL-6, IL 18);
Change from Baseline to Day 15 in plasma levels of ferritin, eosinophil and lymphocyte counts;
Incidence of subjects with treatment related side effects during treatment and up to 30 days after the last treatment dose.

The primary endpoint for efficacy is the response rate of dapansutrile as compared to placebo or active control by Day 15. The primary endpoint of response rate is defined as the proportion of subjects that do not progress to pneumonia or ventilation.

### Example 5. Clinical Trial of Orally Administered Dapansutrile Capsules for the Treatment of Moderate COVID-19 Symptoms

### Methodology

The study is a randomized, double-blinded, placebo-controlled trial to evaluate safety and efficacy of orally administered dapansutrile capsules to mitigate the pulmonary and systemic sequelae associated with early cytokine release syndrome in COVID-19 subjects with confirmed severe acute respiratory syndrome coronavirus 2 (SARS-CoV-2) infection and moderate symptoms.

Approximately 60-100 subjects randomized 1:1 (dapansutrile: placebo) are enrolled.

At the Screening/Baseline/Day 1 Visit, subjects receive the first dose (2000 mg) of dapansutrile or placebo at this visit, and the second dose of dapansutrile (1000 mg) or placebo is taken approximately 12 hours after the first dose. Dapansutrile (2000 mg/day) is continued twice daily (morning and evening doses) through Day 14.

Subjects have blood drawn at the Screening/Baseline/Day 1 Visit, Visit 2/Day 4, Visit 3/Day 8, and Visit 4/Day 15 to assess plasma drug exposure and inflammatory biomarkers, including IL-1β, IL-6, IL-18, IP-10, G CSF, C3a, ferritin, D-dimer, neutrophil count, lymphocyte count, and CRP. Assessment of the subject's COVID-19 symptoms and temperature is also occurred on these days.

Safety and tolerability are evaluated by monitoring the occurrence of adverse effects, vital signs, and clinical safety laboratory test results (chemistry, hematology, and urinalysis) at Screening/Baseline/Visit 1/Day 1, Visit 2/Day 4, Visit 3/Day 8, and Visit 4/Day 15.

All subjects are expected to complete the full 14 days of dosing.

Each subject is asked to maintain two paper diaries at home daily for the first 14 days: a dosing diary and a subject diary. The subject diary is used to record temperature, oxygen levels, COVID-19 symptoms, and overall health. The set of questions used in the subject diary is provided to the subjects at the Screening/Baseline/Day 1 Visit (pre-dose), Day 15, Day 29, and Day 45 visits.

At Day 29 and Day 45 (±3 days), additional assessments of safety and clinical activity occur.

### Main Criteria for Inclusion:

Male and female ≥18 years of age
Positive COVID-19 test ≤5 days prior to randomization
Subjects with moderate COVID-19 who have fever (temperature ≥38° C./100.4° F.) and shortness of breath (with exertion), not requiring oxygen, and meeting the definition of “moderate” as set forth by the May 2020 FDA Guidance for Industry: COVID-19: Developing Drugs and Biological Products for Treatment or Prevention (FDA, 2020), which includes all of the following criteria:
   a. Respiratory rate: ≥20 breaths/minute
   b. SpO₂: >93% on room air
   c. Heart rate: ≥90 beats/minute;
CRP level ≤20 mg/L at Screening/Baseline/Day 1 Visit;
Subject must possess at least one of the following high-risk conditions known to have an underlying increased level of cytokine production:
   1. 70 years or more of age,
   2. Obesity (body mass index [BMI]≥30 kg/m2),
   3. Diabetes (type 1 or 2),
   4. Uncontrolled hypertension,
   5. Known respiratory disease (including asthma or chronic obstructive pulmonary disease [COPD]),
   6. Known heart failure (note: subjects with New York Heart Association Class IV congestive heart failure cannot be enrolled per Exclusion Criterion 4), or
   7. Known coronary disease;

### Main Criteria for Exclusion:

### Main Criteria for Exclusion:

1) Women of childbearing potential, or men whose sexual partner(s) is a woman of childbearing potential, who:
   a. Are or intend to become pregnant during the study;
   b. Are nursing (female subjects only);
   c. Are not using an acceptable, highly effective method of contraception until all follow-up procedures are complete.
2) Evidence of pre-existing or new-onset organ failure;
3) Evidence of moderate concurrent nervous system, renal, endocrine, or gastrointestinal disease, unrelated to COVID-19 as determined by the Investigator (with the exception of those conditions required for enrollment);
4) Evidence of cardiovascular disease with significant arrhythmia, congestive heart failure (New York Heart Association Class IV), unstable angina, uncontrolled hypertension, cor pulmonale, or symptomatic pericardial effusion, not related to COVID-19 as determined by the Investigator (with the exception of those conditions required for enrollment);
5) Required use of vasoactive drug support;
6) History of myocardial infarction in the 6 months prior to the Screening/Baseline/Day 1 Visit;
7) Evidence of current liver disease, not related to COVID-19 as determined by the investigator;
8) History or evidence of active tuberculosis (TB) infection at Screening/Baseline/Day 1 Visit or one of the risk factors for tuberculosis;
9) History of or currently active primary or secondary immunodeficiency;
10) Past or present requirement for oxygen (e.g., nasal cannula, proning, mechanical ventilation and/or supplemental oxygen);
11) Use of any prohibited concomitant medications/therapies, including specifically:
a. use of ibuprofen or diclofenac
b. use of colchicine
c. use of systemic steroids within 30 days of randomization
d. use of janus kinase (JAK) inhibitors
e. use of off-label agents (e.g., hydroxychloroquine, remdesivir, dexamethasone) and biologic and oral anti-cytokine agents (e.g., current treatment with adalimumab, infliximab, etanercept, golimumab, certolizumab pegol, tocilizumab, sarilumab, anakinra, canakinumab, rilonacept, baricitinib, tofacitinib, or upadacitinib);
   Note: During the treatment period, a subject may meet the criteria for a treatment approved by the FDA specifically for COVID-19 (e.g., remdesivir). In this situation the investigator and medical monitor should confer and take the most appropriate decision for the subject.
12) Known history of renal impairment (e.g., calculated glomerular filtration rate [GFR]<45 mL/min);
13) Evidence of malignant disease, or malignancies diagnosed within the previous 5 years;
14) History of infection or known active infection with human immunodeficiency virus (HIV), hepatitis B virus (HBV), or hepatitis C virus (HCV);
15) Any other concomitant medical or psychiatric conditions, diseases, or prior surgeries that, in the opinion of the Investigator, would impair the subject from safely participating in the trial and/or completing protocol requirements;
16) Individuals who have been in a chronic care facility in the past 30 days;
17) Individuals who are incarcerated;
18) Participation in any clinical trial and/or use of any investigational product within the immediate 30-day period prior to the Screening/Baseline/Day 1 Visit.

### Dose and Mode of Administration:

The daily total dose of dapansutrile is 2000 mg by oral capsule administration (250 mg per capsule), with the exception of Day 1, in which dapansutrile is dosed at 3000 mg.

| | Day 1 | | Day 2-14 | |
|---|---|---|---|---|
| | First | Second | Morning | Evening |
| | Dose | Dose | Dose | Dose |
| Treatment | 2000 mg | 1000 mg | 1000 mg | 1000 mg |
| Arm 1 | (8 capsules) | (4 capsules) | (4 capsules) | (4 capsules) |
| (Dapansutrile) | | | | |
| Treatment | 8 capsules | 4 capsules | 4 capsules | 4 capsules |
| Arm 2 | | | | |
| (Placebo) | | | | |

### Clinical Trial Duration:

The trial duration is approximately 45 days for all subjects enrolled, with assessments as follows: Screening/Baseline/Day 1, Day 4 (±1 day), Day 8 (±1 day), Day 15 (±1 day), Day 29 (±3 days), and Day 45 (±3 days).

The study design of Example 2 is shown in FIG. 3 for outpatients or ambulatory care. The study design for inpatients or hospitalized patients is similar to that described in FIG. 3 except patients are confined in the hospital and visits are all on-site visits.

### Primary Objective

To assess the clinical efficacy of dapansutrile versus placebo in subjects presenting with moderate COVID-19 respiratory symptoms and evidence of early cytokine release syndrome.

Primary Efficacy Endpoint: Proportion of subjects with complete resolution of fever symptoms (feeling feverish, chills, shivering and/or sweating) and shortness of breath by Day 15.

### Secondary Objectives

### To Assess:

Clinical safety and tolerability of dapansutrile including frequency, type, and severity of adverse events (AEs) and serious adverse events (SAEs), changes in vital signs, as well as safety laboratory data leading to early discontinuation of treatment, study drug related discontinuation of treatment, or treatment emergent Grade 3 adverse events whether or not related to study drug;
Proportion of subjects who experience clinical resolution of fever symptoms and shortness of breath at the Day 8, Day 29, and Day 45 visits;
Time to clinical improvement in fever symptoms and shortness of breath;
Time to sustained absence of fever, defined as at least 2 days since last temperature measurement of ≥38° C. (100.4° F.);
Proportion of subjects who experience clinical improvement in individual symptoms relevant to COVID 19 (e.g., cough, diarrhea, vomiting);
Time to recovery of each symptom relevant to COVID-19 (e.g., cough, diarrhea, vomiting);
Proportion of subjects requiring hospitalization, supplemental oxygen, or mechanical ventilation or who die before Day 15. Hospitalization is defined as ≥24 hours of acute care;
Proportion of subjects who have an improvement from Baseline in their score by Day 15 on the World Health Organization (WHO) Ordinal Scale for Clinical Improvement;
Improvement in oxygenation over the course of the study and maintenance of this effect;
Immunological and inflammatory biomarkers; C-reactive protein (CRP); hematological parameters, as listed below:
   To assess and compare changes in:
   Alanine aminotransferase (ALT), U/L
   Aspartate aminotransferase (AST), U/L
   Blood glucose, mg/dL
   Erythrocyte Sedimentation Rate (ESR)
   Hemoglobin A1c (HbA1C), %
   Lactate dehydrogenase (LDH), U/L
   Lymphocyte, Absolute count
   Monocyte, Absolute count
   Neutrophils, Absolute count
   Eosinophil Absolute count
   To assess and compare changes in COVID-19-related biomarkers:
   CRP
   D-dimer
   Ferritin
   Fibrinogen
   Partial Thromboplastin Time (PTT) and International Normalized Ratio (INR)
   To assess and compare changes in cytokine levels:
   Interleukin (IL)-1β, IL-6, IL-18, granulocyte colony-stimulating factor (G CSF), interferon-γ-induced protein 10 (IP-10), C3a;
To assess and compare changes in respiratory function:
Heart rate
Oxygen saturation/blood oxygen saturation level (SpO₂)
Respiratory rate.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude the specification.

1. A method for treating a COVID-19 patient with mild or moderate respiratory symptoms, comprising administering to a patient in need thereof an effective amount of dapansutrile.

2. The method according to claim 1, wherein said patient is tested positive with SAR-CoV-2 virus.

3. The method according to claim 1, wherein the method treats cytokine release syndrome and prevents cytokine storm and/or full-blown pneumonitis.

4. The method according to claim 1, wherein the patient has respiratory rate ≥20 breaths/minute, SpO2>93% on room air, and heart rate ≥90 beats/minute.

5. The method according to claim 1, wherein the patient has at least one of the high-risk conditions selected from the group consisting of: at least 70 years old, obesity, diabetes, uncontrolled hypertension, a respiratory disease, heart failure, and a coronary disease.

6. The method according to claim 1, which reduces the progression of lung inflammation in the patient.

7. The method according to claim 1, which reduces the hospitalization and mortality rate of the patient.

8. The method according to claim 1, wherein said dapansutrile is orally administered.

9. A method for preventing or treating macrophage activation syndrome in an infected patient, comprising administering to a patient in need thereof an effective amount of dapansutrile.

10. The method according to claim 9, wherein said patient also has chronic obstructive pulmonary disease (COPD), diabetes, and/or heart disease.

11. The method according to claim 9, wherein said patient is a COVID-19 patient.

12. The method according to claim 9, wherein said patient has mild or moderate respiratory symptoms and the method treats cytokine release syndrome and prevents cytokine storm and/or full-blown pneumonitis.

13. The method according to claim 9, which reduces the progression of lung inflammation in the patient.

14. The method according to claim 9, wherein said dapansutrile is orally administered.


SEQUENCE LISTING (6 sequences)

SEQ ID NO: 1 - DNA, 20 bp, Artificial Sequence
  Synthetic nucleic acid
gaatctcaggcacctttacc

SEQ ID NO: 2 - DNA, 22 bp, Artificial Sequence
  Synthetic nucleic acid
gcagttgtctaattccaacacc

SEQ ID NO: 3 - DNA, 21 bp, Artificial Sequence
  Synthetic nucleic acid
aagtcggcagagatttatcca

SEQ ID NO: 4 - DNA, 20 bp, Artificial Sequence
  Synthetic nucleic acid
gatgtcaacctcagctccag

SEQ ID NO: 5 - DNA, 24 bp, Artificial Sequence
  Synthetic nucleic acid
ctaaacagatgaagtgctccttcc

SEQ ID NO: 6 - DNA, 21 bp, Artificial Sequence
  Synthetic nucleic acid
cacataagcctcgttatccca